(19) United States
(12) Patent Application Publication
(10) Pub. No.: US 2020/0033325 A1
(43) Pub. Date: Jan. 30, 2020

(54) ASSAY FOR PHAGOCYTIC IMMUNE CELL DEFECTS ASSOCIATED WITH AUTISM

(71) Applicant: The Trustees of Columbia University in the City of New York, New York, NY (US)

(72) Inventors: Michele Shirasu-Hiza, New York, NY (US); Julie Canman, New York, NY (US)

(21) Appl. No.: 16/484,272

(22) PCT Filed: Feb. 20, 2018
(86) PCT No.: PCT/US2018/018733

§ 371 Date: Aug. 7, 2019

Related U.S. Application Data
(60) Provisional application No. 62/460,158, filed on Feb. 17, 2017.

(51) Int. Cl. G01N 33/50; G01N 33/68

(52) U.S. Cl. CPC G01N 33/5047; G01N 33/5091; G01N 2333/315; G01N 2333/245; G01N 2800/30; G01N 33/6896

(57) ABSTRACT

This invention is in the field of autism and autism spectrum disorder, and relates to an in vitro assay and method to detect phagocytic immune cell defects associated with autism or susceptibility to autism or autism spectrum disorder.

### STATEMENT OF GOVERNMENT SUPPORT

This invention was made with government support under F31NS080673, awarded by NIH. The government has certain rights in the invention.

### FIELD OF THE INVENTION

This invention is in the field of autism and relates to an in vitro method and assay to detect phagocytic immune cell defects associated with autism or autism spectrum disorder, or susceptibility to autism or autism spectrum disorder.

### BACKGROUND OF THE INVENTION

Autism, one of the autism spectrum disorders (ASDs), is mostly diagnosed clinically using behavioral criteria because few specific biological markers are known for diagnosing the disease. Autism is a neuropsychiatric developmental disorder characterized by impaired communication, both verbal and non-verbal, and reciprocal social interaction. It is also characterized by restricted and stereotyped patterns of interests and activities, as well as the presence of developmental abnormalities by three years of age (Bailey et al., 1996). Autism-associated disorders, diseases or pathologies can comprise any number of metabolic, immune or systemic disorders, including gastrointestinal disorders, epilepsy, congenital malformations or genetic syndromes, anxiety, depression, attention deficit disorder, speech delay, and motor uncoordination.

The most common known monogenic cause of intellectual disability and autism in humans is Fragile X syndrome (Kelleher and Bear, 2008). In Fragile X syndrome, expansion of repeated DNA sequences in the genome induce transcriptional silencing of the highly conserved FMR1 gene and lead to loss of FMR1 protein, an mRNA-binding protein and translational inhibitor that is ubiquitously expressed throughout the body, with a strong enrichment in neurons (Darnell et al., 2011; Jin and Warren, 2000). Loss of FMR1 function in humans and animal models is associated with excessive growth of dendritic spines (Irwin et al., 2001; Pan et al., 2004) and defects in synaptic plasticity (Bear et al., 2004; McBride et al., 2005), symptoms that are also associated with other forms of autism (Hutsler and Zhang, 2010; Tang et al., 2014).

It has become appreciated that increased incidences of autism are strikingly correlated with maternal autoimmune diseases and infection during pregnancy (Estes and McAllister, 2015; Gesundheit et al., 2013). As a result, neurological symptoms of autism have been proposed to arise from defects in immune system function, perhaps due to prenatal immune challenge (Mead and Ashwood, 2015). Consistent with this idea, Fragile X syndrome is also associated with altered immune system functions, including elevated proinflammatory cytokine levels in the blood and gastrointestinal inflammation (Estes and McAllister, 2015; Samsam et al., 2014). However, it remains unclear whether defects in immune system function actively contribute to the progression of Fragile X syndrome or if they arise independently of the neuronal defects in this disorder. Moreover, the precise defects in other cellular immune functions in Fragile X syndrome models have not been widely investigated.

An essential conserved function of specialized immune cells in Drosophila and mammals is phagocytosis, or the engulfment of extracellular material generated by foreign pathogens and receptors at the cell surface (e.g. CED-1/Draper), rearrangement of the cytoskeleton, and internalization of target material into a subcellular vesicle called the phagosome. Phagosomes undergo subsequent maturation through fusion with endosomes and lysosomes (Freeman and Grinstein, 2014). Phagocytosis by immune cells is a multistep process that requires an external signal (e.g. pathogenic bacteria), and activation of phagocytic acidic phagolysosome, which degrades the engulfed material. Drosophila have several types of phagocytic cells, including primitive macrophages (or hemocytes) in the circulatory system and phagocytic glia in the brain, which play critical roles in defense against bacterial pathogens such as S. pneumoniae and S. marcescens, the scavenging of dead cellular debris, and active pruning of neuronal axons and dendrites during development.

Recent research has implicated misregulation of astrocytes, or a type of vertebrate glia, in mouse models of Fragile X syndrome (Jacobs and Doering, 2010; Pacey et al., 2015) and Rett syndrome, another cause of autism spectrum disorder in humans (Lioy et al., 2011). For example, astrocytes from Fmr1 mutant mice co-cultured in vitro with neurons from wild type or Fmr1 mutant mice caused excessive dendritic branching, a pathological morphology observed in Fragile X syndrome patients. Wild type astrocytes co-cultured in vitro with neurons from wild type or Fmr1 mutant mice did not cause this phenotype (Jacobs and Doering, 2010). Other common neuroanatomical features of Fragile X syndrome patients and animal models include increased dendritic spine density and decreased axonal pruning (Irwin et al., 2001; Lee et al., 2003; Pfeiffer and Huber, 2009; Tessier and Broadie, 2008). Both of these defects are also associated with defects in glia-mediated phagocytosis (Schafer and Stevens, 2013). Though glia-mediated phagocytosis is required for neuronal structure and function (Blank and Prinz, 2012; Chung and Barres, 2012; Logan et al., 2012), defects in phagocytosis by glia or other immune cells have not previously been demonstrated in any model of Fragile X syndrome.

The prevalence of autism in the United States is about 1 in 91 births, largely due to changes in diagnostic practices, services, and public awareness. Autism is growing at the fastest pace of any developmental disability (Fombonne 2003). Care and treatment of autism costs the United States healthcare system about $90 billion annually. Early detection and intervention can result in reducing life-long costs. At present, few tools outside psychiatric evaluation are available for diagnosing autism. Thus, there is a need for a non-invasive but accurate test for diagnosing autism.

### SUMMARY OF THE INVENTION

The current invention is based upon the discovery that Fmr1 mutant Drosophila have decreased phagocytosis in their hemocytes as well as in their glial cells and Fmr1 knock out mice have defects in their glial-dependent synaptic pruning, which requires glia-mediated phagocytosis. Fmr1 mutant flies and mice are recognized models of Fragile X syndrome, a form of autism, which in turn is a model for other forms of human autism. Without being bound by any theory, it is believed that the decreased phagocytosis in the glial cells of the mutants is at least partially responsible for their phenotype, i.e., model of Fragile X syndrome. This decreased phagocytosis in their glial cells in the flies correlates with decreased phagocytosis in their hemocytes, which are the equivalent to phagocytic immune cells found in the blood of mammals. Defects in glial phagocytosis in the Drosophila model leads to neuroanatomical pruning defects similar to those observed in many types of autism spectrum disorder. Additionally, there is a correlation between the defects in immune cells in the blood and brain of humans (Yin et al., 2017), thus, providing the current invention: an easy, non-invasive in vitro assay for the detection of phagocytic cells in a sample, e.g., blood, from patients to detect for autism or a predisposition for autism.

Thus an embodiment of the current invention is an in vitro method of detecting autism or a predisposition to autism in a subject comprising: isolating phagocytic immune cells from a sample from the subject; contacting or incubating the phagocytic immune cells with bioreactive particles in an amount and for a period of time sufficient for phagocytic immune cells to phagocytose the bioreactive particles; detecting the number or quantity of phagocytic immune cells which phagocytosed the bioreactive particles after the period of time; comparing the number or quantity of the phagocytic immune cells in the sample which phagocytosed the bioreactive particles after the period of time to a reference value of the number or quantity of phagocytic immune cells which would phagocytose the bioreactive particles in the same period of time; and detecting autism or a predisposition to autism when the number or quantity of phagocytic immune cells in the sample which phagocytosed the bioreactive particles after the period of time is less than the reference value.

A further embodiment of the current invention is an in vitro method of detecting autism or a predisposition to autism in a subject comprising: isolating phagocytic immune cells from a sample from the subject; contacting or incubating the phagocytic immune cells with bioreactive particles in an amount and for a period of time sufficient for phagocytic immune cells to phagocytose the bioreactive particles; detecting the number or quantity of the bioreactive particles that are not phagocytosed after the period of time; comparing the quantity of the bioreactive particles in the sample not phagocytosed after the period of time to a reference value of the number or quantity of bioreactive particles not phagocytosed in the same period of time; and detecting autism or a predisposition to autism when the number or quantity of bioreactive particles in the sample not phagocytosed after the period of time is greater than the reference value.

In some embodiments, the subject is human and in further embodiments, the subject is a human child under the age of three years old. In some embodiments, the human child is between the ages of three and five years old, and in further embodiments the human child is over five years old. In some embodiments, the subject is suspected of having autism based upon a behavioral and clinical examination. In some embodiments, the subject is not suspected of having autism.

In some embodiments, the sample is blood.

In some embodiments, the bioreactive particles are bacteria. In some embodiments, the bacteria are heat killed or otherwise inactivated. In some embodiments, the bacteria are E. coli, Streptococcus pneumoniae or Serratia marcescens.

In further embodiments, the bioreactive particles are cells, portions of cells, or cell fragments. In some embodiments, the cells are apoptotic.

In some embodiments, the bioreactive particles are synthetic, and in some embodiments the bioreactive particles are latex beads. In yet another embodiment, the bioreactive particles are Zymosan (Saccharomyces cerevisiae), prepared from yeast cell wall and consisting of protein:carbohydrate complexes.

In some embodiments, the bioreactive particles comprise a detectable label, such as fluorescence.

In some embodiments, the phagocytic immune cells are macrophages or other phagocytic leukocytes.

In some embodiments, the reference value is a known number or quantity of bioreactive particles, e.g., bacteria, phagocytosed by immune cells in a sample, e.g., blood, from a healthy control subject. In some embodiments, the reference value is obtained by performing the same method or assay on a sample from a healthy control subject.

The current invention also provides kits for the detection of autism or the predisposition of autism which can comprise: reagents for obtaining a sample; reagents for isolating, plating and culturing phagocytic immune cells; bioreactive particles for inducing phagocytosis; and instructions for use including the amount of bioreactive particles to contact or incubate with the cells and the amount of time to contact or incubate the bioreactive particles with the cells, and a reference value for comparison. The bioreactive particles of the kit can be optionally labeled for detection and/or measuring.

### BRIEF DESCRIPTION OF THE FIGURES

FIG. 1 contains the results showing that immunity against infection was defective in Fmr1 mutants. FIG. 1A shows the survival curve of wild type controls and Fmr1 mutants after infection by S. pneumoniae (WT, n=59; Fmr1, n=53; p<0.0001). FIG. 1B shows the survival curve of wild type controls and Fmr1 mutants after infection by S. marcescens (WT, n=60; Fmr1, n=61; p<0.0001). These figures show that the Fmr1 mutants were highly sensitive to infection by these bacterial pathogens. FIG. 1C shows the bacterial load of wild type controls (left hand side of each time period) and Fmr1 mutants (right hand side of each time period) 3 and 18 hours post infection with S. pneumoniae (n=6 each genotype, each time point; p<0.01). FIG. 1D shows the bacterial load of wild type controls (left hand side of each time period) and Fmr1 mutants (right hand side of each time period) 24 hours post infection with S. marcescens (n=6 each genotype, each time point; p<0.01). These figures show relative to wild type controls, Fmr1 mutants were less able to kill and clear these pathogens, as shown by higher bacterial loads.

FIG. 2 shows that phagocytosis by immune cells in circulation was defective in Fmr1 mutants. FIG. 2A is a graph of the quantification of overall phagocytic activity in the thorax of wild type controls (n=6), and Fmr1 mutants (n=6) as shown by internalization of dead S. aureus labeled with pHrodo, a pH-sensitive dye, p<0.01. Fmr1 mutants exhibited reduced total phagocytosis by immune blood cells (hemocytes) relative to wild type. FIG. 2B are graphs of the quantification of pHrodo fluorescence (WT, n=10; Fmr1, n=9; p<0.01), hemocyte-specific GFP (hemocytes genetically labeled by hemlΔ-Gal4 driving UAS-GFP expression) (p>0.05), and phagocytic activity normalized to hemocyte-specific GFP hemocytes (p<0.01) in both control wild type flies and Fmr1 mutants. Fmr1 mutants exhibited less phagocytic activity per hemocyte. FIG. 2C is a graph of the quantification of hemocyte engulfment of S. aureus as measured by injection of Alexa 594-labeled dead S. aureus, followed by Trypan Blue quench (controls, n=10; Fmr1 mutants, n=10; p<0.0001), hemocyte-specific GFP (hemocytes genetically labeled by hemlΔ-Gal4 driving UAS-GFP expression) (p>0.05), and phagocytic activity normalized to hemocyte-specific GFP hemocytes (p<0.01) in both control wild type flies and Fmr1 mutants. FIG. 2D shows the number of phagocytic hemocytes in Fmr1 mutants and Fmr1 mutants rescued by an exogenous Fmr1 transgene expression construct. ****p<0.0001, **p<0.01, n.s.: p>0.05. p-values obtained by Mann-Whitney test. Mean±SEM shown, variance shown by scatter plot. FIG. 3 shows that Fmr1 functions cell autonomously in circulating immune cell phagocytosis. FIG. 3A shows the levels of phagocytosed pHrodo S. aureus bacteria quantified per fly for each genotype (flies containing hemlΔ-Gal4 driver alone, flies containing UAS-Fmr1 RNAi alone, and flies containing hemlΔ-Gal4 driver expressing UAS-Fmr1 RNAi) (n=17-31 flies per genotype, p<0.0001 for hemlΔ-Gal4 driver expressing UAS-Fmr1 RNAi relative to both controls). FIG. 3B shows the hemocyte number in the thorax was not changed by hemlΔ driver expression of Fmr1 RNAi. (n=15-16 flies per genotype, p=0.9335). FIG. 3C is a survival curve of flies containing hemlΔ-Gal4 driver alone, flies containing UAS-Fmr1 RNAi alone, and flies containing hemlΔ-Gal4 driver expressing UAS-Fmr1 RNAi after infection by S. pneumoniae showing that relative to hemlΔ-Gal4 driver alone (grey) or Fmr1 RNAi alone (pink), hemlΔ-Gal4 driver expressing Fmr1 RNAi flies were sensitive to infection by S. pneumoniae (hemlΔ-Gal4 driver alone, n=80; Fmr1 RNAi alone, n=76; hemlΔ-driver expressing Fmr1 RNAi, n=75; p<0.0001). p-values obtained by: FIG. 3A—ANOVA followed by Tukey's multiple comparison test; FIG. 3B—unpaired t-test; FIG. 3C—log-rank analysis; mean±SEM shown, variance shown by scatter plot.

FIG. 4 shows that glial response to axonal injury was delayed in adult Fmr1 mutants. FIG. 4A shows sample fluorescence images and quantification of clearance of GFP-labeled neurons in control wild type (left hand side, n=11) and Fmr1 mutants (right hand side, n=10) 18 hours after axotomy, p<0.05. Fmr1 mutants showed reduced clearance of GFP+ olfactory neurons relative to controls. GFP intensity was not different between unwounded controls (Ctrl n=12, Fmr1 n=14), p>0.05. FIG. 4B shows quantification of Draper-expressing glia in wild type controls (left hand side, n=20), and Fmr1 mutants (right hand side, n=23) at the glomeruli 18 hours after axotomy p<0.0001. Fmr1 mutants showed reduced levels of Draper-expressing glia at the glomeruli 18 hours after axotomy relative to controls. Draper levels were not different between unwounded controls (Ctrl n=24, Fmr1 n=18), p>0.05. FIG. 4C shows graphs of the total fluorescence intensity of GFP-labeled neurons and Draper over time showing the time course of neuronal clearing and Draper levels after axon wounding. Controls are the left hand bars and the Fmr1 mutants the right hand bars for each time period. Fmr1 mutants exhibited delays but not total inhibition of axonal clearance and Draper expression in response to neuronal wounding relative to controls (n=9-14 for each genotype for each time point). ****p<0.0001, ***p<0.001, **p<0.01, *p<0.05, n.s.: p>0.05. p-values were obtained by unpaired, two-tailed t-test with Welch's correction except when normality test was not passed, when Mann-Whitney was used; mean±SEM shown, variance shown by scatter plot. Scale bars=10 μm.

FIG. 5 shows that Fmr1 mutants exhibited delayed pruning by glia during development. FIG. 5A are representative images of anti-Fasciclin II staining of the developing mushroom body in wild type and Fmr1 mutants at 6, 12, 18, and 24 hours after pupation showing delayed pruning of γ-neurons (γ-MB) by astrocytes in Fmr1 mutants relative to wild type. FIG. 5B is a graph showing the distribution of morphological phenotypes over time. This figure shows an increased percentage of Fmr1 mutants with partial pruning phenotypes at 12 and 18 hours after pupation relative to wild type (averages of three trials, total n=25-50 per genotype per time point). FIG. 5C shows, for time points after pupation, the percentages of structures that are fully pruned. At 12 hours after pupation, wild type flies (left hand bars) exhibited a significantly higher percentage of structures (75%) with the fully pruned morphology than Fmr1 mutants (right hand bars, 37%), n=3 trials, total WT n=50, total Fmr1 mutants n=33, p<0.05. p-value obtained by unpaired, two-tailed t-test with Welch's correction; mean±SD shown. Scale bar=10 μm.

FIG. 6 shows a defect in the synaptic refinement of young adult Fmr1 knock out mice. FIG. 6A are representative images of the neurons in the dorsal lateral geniculate (dLGN) in wild type and Fmr1 knock out mice at postnatal day 40. FIG. 6B is a graph of percent ipsilateral segregation versus collateral threshold in wild type (n=6) and Fmr1 knock out mice (n=4).

### DETAILED DESCRIPTION OF THE INVENTION

### Definitions

The terms used in this specification generally have their ordinary meanings in the art, within the context of this invention and the specific context where each term is used. Certain terms are discussed below, or elsewhere in the specification, to provide additional guidance to the practitioner in describing the methods of the invention and how to use them. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of the other synonyms. The use of examples anywhere in the specification, including examples of any terms discussed herein, is illustrative only, and in no way limits the scope and meaning of the invention or any exemplified term. Likewise, the invention is not limited to its preferred embodiments.

As used herein, the term “sample” means any substance containing or presumed to contain cells, in particular immune cells, more particularly phagocytic immune cells (e.g., macrophages). The sample can be a sample of tissue or fluid isolated from a subject including but not limited to, plasma, serum, whole blood, spinal fluid, semen, amniotic fluid, lymph fluid, synovial fluid, urine, tears, blood cells, organs, and tissue. A preferred sample is blood.

As used herein, the term “subject” means any organism including, without limitation, a mammal such as a mouse, a rat, a dog, a guinea pig, a ferret, a rabbit and a primate. In the preferred embodiment, the subject is a human being, a pet or livestock animal.

The term “patient” as used in this application means a human subject.

The term “detection”, “detect”, “detecting” and the like as used herein means as used herein means to discover the presence or existence of.

The term “reference value” as used herein means a known number or quantity of bioreactive particles phagocytosed by phagocytic immune cells from a healthy control. The reference value can also be the number or quantity of bioreactive particles not phagocytosed by phagocytic immune cells from a healthy control.

The terms “healthy control” as used herein is a human subject who is not suffering from autism or at risk for autism. In addition, a healthy control can be aged matched to the subject being tested, and not suffering from other diseases or conditions that would affect his or her immune response.

The term “about” or “approximately” means within an acceptable error range for the particular value as determined by one of ordinary skill in the art, which will depend in part on how the value is measured or determined, i.e., the limitations of the measurement system, i.e., the degree of precision required for a particular purpose, such as a pharmaceutical formulation. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

The results herein were obtained using Fmr1 Drosophila mutants, a well-established Drosophila model of Fragile X syndrome (Coffee et al., 2010; Wan et al., 2000), which in turn is a model for autism, as well as a mouse model of Fragile X syndrome, a Fmr1 knock out mouse. First it was found that Fmr1 mutant flies were highly sensitive to infection by two specific bacterial pathogens, S. pneumoniae and S. marcescens, the defense against which requires phagocytosis by hemocytes, which are the equivalent of phagocytic immune cells in mammals. It was further found that hemocytes in Fmr1 mutant flies exhibited reduced bacterial engulfment, an early step in phagocytosis. In addition, it was demonstrated that Fmr1 mutant flies also exhibited phagocytic defects in immune cells in the brain (glial). Fmr1 mutant flies exhibited delays in two different phagocytosis dependent processes: axonal clearance after neuronal wounding in the adult brain; and pruning of neuronal processes during development of the mushroom body, a brain structure required for learning and memory. It was further found that delayed axonal clearance in the adult was associated with a delay in recruitment of activated, phagocytic glia to the site of wounded neurons. Because glia-mediated phagocytosis is critical in shaping neuronal structure and function (Blank and Prinz, 2012; Chung and Barres, 2012; Logan et al., 2012), these results show that defects in phagocytic glia contribute to the neurological symptoms of Fragile X syndrome.

Additionally, to determine if there is a defect in phagocytosis by brain immune cells in a mammalian (mouse) model of Fragile X syndrome, the refinement of synapses in the visual system (retinogeniculate system) of young adult mice was examined. Synaptic refinement in the retinogeniculate system has previously been shown by others to require phagocytosis by glia and is currently one of the only and certainly the best system to date for examining glia-mediated phagocytosis in the brain. As shown herein, at postnatal day 40 (young adult mice), a time point when such refinement is usually complete, Fmr1 knock out mice exhibited significantly decreased refinement relative to wild type littermates. These data are the first direct, in vivo evidence of a synaptic elimination decrease in a mammalian model of Fragile X syndrome, defects which are consistent with glia-mediated phagocytosis defects.

These results provide the first direct in vivo demonstration of defects in immune cell-mediated and glial-mediated phagocytosis in a both a Drosophila and mammalian model of Fragile X syndrome. Phagocytic defects could also contribute to the neurological and immunological symptoms associated with Fragile X syndrome in human patients as well as contribute to other types of autism and autism spectrum disorder. While early detection of autism spectrum disorder has been shown to be critical for effective therapeutic intervention, autism spectrum disorders that are not attributed to monogenic causes such as Fragile X syndrome or Rett syndrome can be extremely difficult to diagnose at a young age.

The neuroanatomical pruning defects common to many types of autism spectrum disorder as shown herein to be due to defects that manifest both in phagocytic immune cells of the brain and blood, as well as the known correlation between the defects in immune cells in the blood and brain, provide a strategy for an easy, non-invasive in vitro method and assay for the detection of phagocytic cells in a sample from young patients to detect autism or a predisposition for autism.

Thus, the invention provides methods to detect whether a subject is at risk of developing autism or an autism spectrum disorder (ASD), or suffers from autism or an ASD, wherein the disease is manifested by a decrease of phagocytic immune cells, both in the brain and blood.

Subjects can include humans, who are three years of age or under, who have been diagnosed with autism or ASD based upon clinical and behavioral symptoms. The subject can also be considered at risk for autism or ASD based upon for instance, a sibling with the disorder or other familial history. The human subject can also be between the ages of three and five years old, or older than five years old. In a further embodiment, because the current invention is a noninvasive accurate method for the detection of autism and/or ASD, or the risk of autism and/or ASD, the subject can be any human age three and younger, or age three or older, including those with no symptoms or risks of autism and ASD.

Subject with autism, as well as ASD, can display some core symptoms in the areas of a) social interactions and relationships, b) verbal and non-verbal communication, and c) physical activity, play, and physical behavior. For example, symptoms related to social interactions and relationships can include but are not limited to the inability to establish friendships with children the same age, lack of empathy, and the inability to develop nonverbal communicative skills (for example, eye-to-eye gazing, facial expressions, and body posture). For example, symptoms related to verbal and nonverbal communication comprise delay in learning to talk, inability to learn to talk, failure to initiate or maintain a conversation, failure to interpret or understand implied meaning of words, and repetitive use of language. For example, symptoms related to physical activity, play, and physical behavior can include but are not limited to unusual focus on pieces or parts of an object, such as a toy, a preoccupation with certain topics, a need for routines and rituals, and stereotyped behaviors (for example, body rocking and hand flapping).

A subject with any of these symptoms would be a candidate for testing with the method of the current invention.

In certain embodiments of the invention, the method comprises: isolating phagocytic immune cells from a sample from a subject; contacting or incubating the phagocytic immune cells with bioreactive particles in an amount and for a period of time sufficient to allow phagocytic immune cells to phagocytose the bioreactive particles; detecting the number or quantity of the phagocytic immune cells which phagocytosed the bioreactive particles after the period of time; comparing the number or quantity the phagocytic immune cells which phagocytosed the bioreactive particles to a reference value of the number or quantity of phagocytic immune cells which phagocytosed bioreactive particles for the same period of time; and detecting autism or autism spectrum disorder, or a predisposition to autism or autism spectrum disorder if the number or quantity of phagocytic immune cells which phagocytosed the bioreactive particles is less than the reference value.

In other embodiment of the invention, the method comprises: isolating phagocytic immune cells from a sample from a subject; contacting or incubating the phagocytic immune cells with bioreactive particles in an amount and for a period of time sufficient to allow phagocytic immune cells to phagocytose the bioreactive particles; detecting the number or quantity of bioreactive particles which are not phagocytosed after the period of time; comparing the number or quantity of bioreactive particles which are not phagocytosed after the period of time to a reference value of the number or quantity of bioreactive particles which are not phagocytosed after the same period of time; and detecting autism or autism spectrum disorder, or a predisposition to autism or autism spectrum disorder if the number or quantity of bioreactive particles is greater than the reference value.

Various techniques known in the art can be used to detect cells that have phagocytosed the bioreactive particles or to detect unphagocytosed bioreactive particles in a sample from a subject.

While any sample that would contain phagocytic immune cells (e.g., macrophages) can be used, a preferred sample is blood.

The cells are isolated from the sample by any method known in the art and are optionally plated and cultured, optionally in welled assay dishes or plates, e.g., 24-well, 48-well, and 96-well. Cells should generally be in a concentration of about 1-5×10⁵ cells/ml.

The cells are then contacted or incubated with bioreactive particles, which is any particle that would elicit a response from the cells. Bioreactive particles can be bacteria, including but not limited to, E. coli, S. pneumoniae and S. marcescens. Bioreactive particles can be yeast. Bioreactive particles can also be cells, or portions of cells, or cell fragments. In some embodiments the cells are apoptotic. Bioreactive particles can be Zymosan. Additionally, bioreactive particles can also be synthetic and would include but not limited to latex beads, latex beads coated with antibodies, fluorescent dyes or proteins, and autofluorescent latex beads.

The concentration of bioreactive particles contacted or incubated with the cells would be within the skill of the art, and can range from about 5 mg/ml to 100 mg/ml. More preferably, the concentration can range from about 5 mg/ml to about 80 mg/ml, or from about 10 mg/ml to about 80 mg/ml, or from about 10 mg/ml to about 70 mg/ml, or from about 10 mg/ml to about 60 mg/ml, or from about 10 mg/ml to about 50 mg/ml, or from about 10 mg/ml to about 40 mg/ml, or from about 10 mg/ml to about 30 mg/ml, or from about 15 mg/ml to about 80 mg/ml, or from about 15 mg/ml to about 70 mg/ml, or from about 15 mg/ml to about 60 mg/ml, or from about 15 mg/ml to about 50 mg/ml, or from about 15 mg/ml to about 40 mg/ml, or from about 15 mg/ml to about 30 mg/ml. In one embodiment, the concentration used would be about 20 mg/ml.

The bioreactive particles can optionally be labeled for detection and/or measurement of phagocytosis. These labels include any known in the art and include but are not limited to, fluorescein isothiocyanate (FITC), carboxyfluorescein succinimidyl ester (CFSE), pHrodo® fluorescence, and various AlexaFluor® dyes.

Additionally, labeled bioreactive particles such as Zymosan labeled with FITC and S. aureus labeled with pHrodo® are commercially available from ThermoFisher Scientific.

The cells that have phagocytosed the labeled reactive particles can be visualized by any method known in the art including but not limited to the use of flow cytometry, fluorescence microscopy, and spectrophotometer. The bioreactive particles that have not been phagocytosed can also be visualized by any method known in the art including but not limited to the use of flow cytometry, fluorescence microscopy, and spectrophotometer.

The bioreactive particles are incubated or contacted with the cells for a period of time that would allow phagocytic cells to phagocytose the bioreactive particles, and ranges from about 30 minutes to about four hours, or from about 30 minutes to about three hours, or from about 30 minutes to about two hours.

The method includes optional steps of stopping the phagocytic reaction and washing and/or blocking unphagocytosed bioreactive particles from the wells or plates.

A reference value of either the number or quantity of phagocytic immune cells which phagocytosed bioreactive particles for the same period of time, or the number or quantity of bioreactive particles which are not phagocytosed after the period of time, can be obtained by performing the same method or assay using a sample from a healthy control, preferably one age matched with the subject. A reference value can also be obtained from literature.

Commercially available assays for phagocytosis are available from various suppliers, including ThermoFisher Scientific, Cayman Chemical, Cell Biolabs, Essen Bioscience, and BioVision.

### Kits

It is contemplated that all of the methods and assays disclosed herein can be in kit form for use by a health care provider and/or a diagnostic laboratory.

Such kits would include reagents for isolating and purifying phagocytic cells from samples from subjects, reagents for performing the methods and assays including bioreactive particles and detection of the bioreactive particles, instructions for use, and in particular, reference values or the means for obtaining reference values from a healthy control sample for comparison.

### EXAMPLES

The present invention may be better understood by reference to the following non-limiting examples, which are presented in order to more fully illustrate the preferred embodiments of the invention. They should in no way be construed to limit the broad scope of the invention.

### Example 1—Materials and Methods for Examples 2-7

### Fly Lines

Fmr1 trans-heterozygous null mutants were generated by crossing two heterozygous mutant lines, each containing a well-characterized Fmr1 null mutation, Fmr1^{Δ50M} (from D. Zarnescu (Zarnescu et al., 2005)) and Fmr1³ (from Tom Jongens (Wan et al., 2000)). Both Fmr1 mutant lines were outcrossed to their wild type controls (Oregon R and iso31b, respectively) for at least six generations. For all experiments, wild type controls were generated by crossing Oregon R and iso31b lines and collecting the heterozygous progeny. Hemocytes were labeled with GFP using a hemlΔ-Gal4 construct paired with UAS-GFP. For hemocyte-specific RNAi of dFMR1, the hemlΔ-Gal4 driver was used to drive expression of UAS-Fmr1-RNAi (VDRC TID 8933). The hemlΔ-Gal4 and UAS-Fmr1-RNAi constructs were outcrossed to W1118 CS for 10 and 6 generations, respectively, and W1118 CS flies were used as controls. Olfactory neurons were labeled using the OR85e::mCD8-GFP construct (from Marc Freeman (Doherty et al., 2009)). Ensheathing glia were labeled with an mz0709-Gal4 construct (from Marc Freeman (Doherty et al., 2009)) paired with UAS-RFP. All transgenic constructs were also outcrossed for 6-8 generations with appropriate wild type control strains to maintain proper genetic backgrounds for experimental use.

### Bacterial Strains

Bacterial infections were performed with two types of bacteria: Streptococcus pneumoniae strain SP1, a streptomycin-resistant variant of D39 from Stan Falkow (Joyce et al., 2004); and Serratia marcescens strain DB1140 from Man Wah Tan (Flyg and Xanthopoulos, 1983).

### Fly Rearing Conditions

Flies were raised at 25° C., 55% humidity on yeasted, low-glutamate food in a 12 h: 12 h light:dark cycle (Chang et al., 2008). Flies collected for survival and hemocyte phagocytosis experiments were maintained on standard dextrose food. The recipe for standard dextrose food is as follows: 38 g/L cornmeal, 20.5 g/L yeast, 85.6 g/L dextrose, and 7.1 g/L agar. Infection experiments were performed with age-matched male flies, 5-7 days post-eclosion. Glial phagocytosis experiments were performed with age-matched females 5-10 days post-eclosion that were maintained on low-glutamate food before and after maxillary palp excision.

### Statistical Analysis

Statistical analyses were performed using GraphPad Prism. When comparing two groups of quantitative data, unpaired, two-tailed t-test with Welch's correction was performed if data showed a normal distribution (determined using D'Agostino & Pearson omnibus normality test) and Mann-Whitney test if data distribution was non-normal. Survival data was analyzed using log-rank analysis.

### Infections

Infection experiments were performed as previously described (Stone et al., 2012). Briefly, flies were anesthetized on CO₂ pads and injected using a custom microinjector (MINJ-Fly, Tritech) and glass capillary needles pulled with a Sutter Instrument P-30. 50 nL of liquid were injected into each fly, calibrated by measuring the diameter of the expelled drop under oil. S. pneumoniae cultures were grown to an OD₆₀₀ of 0.40 at 37° C. in shaking BHI and frozen in 5% glycerol at −80° C. For infection, bacteria were pelleted upon thawing, supernatant removed, resuspended in fresh BHI, and diluted to a final OD₆₀₀ of 0.10-0.12 for injection. S. marcescens was grown in shaking BHI overnight at 37° C. and diluted to OD₆₀₀ ranging from 0.1 to 0.6 for injection into flies. After injection, flies were incubated at 29° C. in a 12 h:12 h light:dark cycle for the duration of the infection.

### Survival Assays

Between 60 and 85 flies per genotype per condition were assayed for each survival curve and placed in three vials of standard dextrose food with approximately 20 flies each. In each experiment, 20-40 flies of each line were also injected with sterile media as a control for death by wounding. Survival proportions were assayed by counting the number of dead flies at various time points post-infection. Data was converted to Kaplan-Meier format using custom Excel-based software called Count the Dead (from Joel Shirasu-Hiza (Stone et al., 2012)). Survival curves were plotted as Kaplan-Meier plots and statistical significance is tested using log-rank analysis with GraphPad Prism software. All experiments were performed at least three times and yielded similar results.

### Bacterial Load Quantitation

Six individual flies were collected at each time point following microbial challenge. These flies were homogenized, diluted serially and plated on tryptic soy blood agar plates (S. pneumoniae) or LB agar plates (S. marcescens). Statistical significance was determined using unpaired, two-tailed t-tests for 0-hour time points and non-parametric Mann-Whitney tests for all other time points to account for exponential growth. All experiments were performed at least three times and yielded similar results.

### Assay of Phagocytosis by Immune Blood Cells

Male flies 5-7 days old were injected with 50 nL of 20 mg/mL pHrodo-labeled S. aureus in PBS (Molecular Probes, A10010) or Alexa 594-labeled S. aureus in PBS (Molecular Probes, S23372). The flies were allowed to phagocytose the particles for 35-45 minutes. Using a thin layer of Loctite superglue, the dorsal surfaces of the flies were glued onto coverslips; for experiments using Alexa 594-labeled bacteria, non-phagocytosed bacteria were quenched by Trypan blue injection into the circulating hemolymph. Fluorescence images were taken of the dorsal surface using epifluorescent illumination with a Nikon Eclipse E800 microscope fitted with a Photometrics Cool Snaps HQ2 camera with 10× or 20× objectives. Images were captured and quantified with Nikon Elements software. To quantify, all of the images within an experiment were thresholded using the same pixel intensity value to define ROIs; the sum and average intensities of the ROIs were then calculated. Experiments were repeated three times with 6-14 flies for each treatment. Assays examining total phagocyte number were performed using flies expressing a UAS-GFP construct with the hemocyte-specific promoter hemlΔ-Gal4. Statistical significance was determined using unpaired, two-tailed t-tests.

### Glial Phagocytosis Assay and Immunohistochemistry

Glial phagocytosis assays were conducted as previously described (MacDonald et al., 2006) with some modifications. The maxillary palps of age-matched 5- to 10-day-old OR85e::mCD8-GFP (from Marc Freeman); Fmr1^{3/Δ50M} mutants and wild-type controls were excised to sever the olfactory neurons. Flies were collected and decapitated at various time points after wounding, and the fly heads were fixed for 40 minutes at room temperature in 4% paraformaldehyde in PBS+0.1% Triton X-100 (PTX). Fly heads were washed five times in PTX and brains were dissected in ice-cold PTX. Brains were blocked in 4% normal donkey serum (NDS) in PTX and incubated in primary antibodies at 4° C. overnight. Primary antibody was diluted in PTX with 2% NDS. The following primary antibodies and dilutions were used: rabbit anti-Draper (from Marc Freeman, 1:500 (MacDonald et al., 2006)); chicken anti-GFP (Abcam 13970, 1:1000); mouse anti-RFP (Abcam 65856, 1:500). Brains were then washed five times over the course of 1 hour at room temperature in PTX and incubated in secondary antibodies at 4° C. overnight. Secondary antibodies were diluted in PTX with 2% NDS. The following secondary antibodies and dilutions were used in this study: Rhodamine Red-X-conjugated donkey anti-rabbit IgG (Jackson Immunoresearch 711-295-152, 1:200), Alexa 488-conjugated donkey anti-chicken IgY (IgG) (Jackson Immunoresearch 703-545-155, 1:200), and Rhodamine Red-X-conjugated donkey anti-mouse IgG (Jackson Immunoresearch 715-295-151, 1:200. Brains were washed and mounted onto coverslips that had been coated with poly-L-lysine (Advanced BioMatrix 5048) and Kodak Photo Flo 200 (0.36%, 1464510), dehydrated by incubating in increasing concentrations of ethanol for 5 minutes (30%, 50%, 75%, 95%, 100%, 100%), and incubated in two different solutions of 100% xylenes for 10 minutes each. Coverslips were mounted onto slides with DPX and dried overnight before imaging. Brains were imaged using a Zeiss LSM 510 Meta with Axiolmager Z1 upright confocal microscope using 488, 561, and 633 nm lasers. Images were taken with a PlanNeoFL 40×/1.3 lens, and the Zeiss LSM confocal software was used for 3D reconstruction. Fiji (ImageJ) was used to quantify the total fluorescence intensity of the three middle slices in a region traced around each glomerulus, normalizing to background fluorescence for each sample. For the glial extension assay, fluorescence of ensheathing glia and Draper was quantified using a standard circular region of interest placed over the glomerulus, normalizing to background fluorescence for each sample. All experiments were performed at least three times and yielded similar results. Statistical significance was determined using unpaired, two-tailed t-tests.

### Developmental Glial Phagocytosis Assay

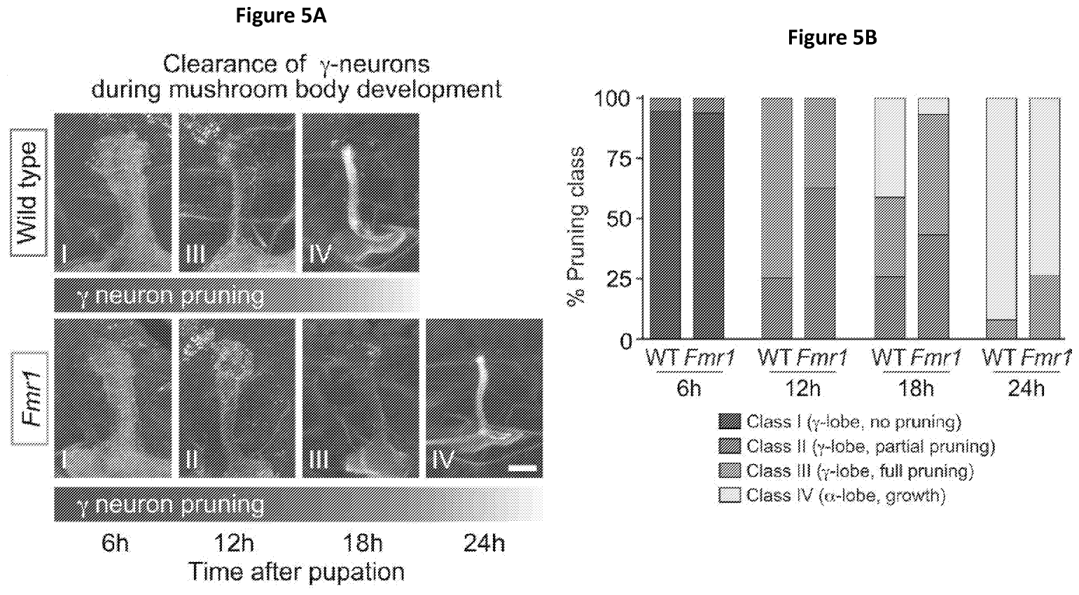

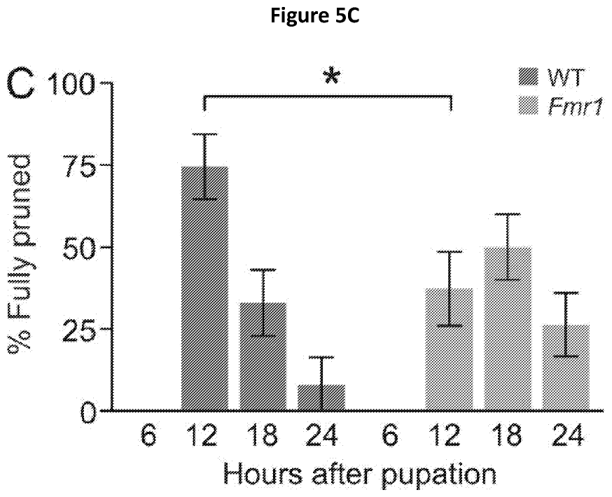

Fmr1^{3/Δ50M} mutants and wild type controls were raised as above and collected at various time points following pupal formation. The pupae were fixed in two 15-minute washes in 4% paraformaldehyde in PTX. They were then washed, stained, and imaged as above. Antibodies used were mouse anti-Fasciclin II (DSHB 1D4, 1:5) and Alexa 488-conjugated donkey antimouse IgG (Jackson Immunoresearch 715-545-151, 1:200). Maximum intensity projections were generated and images blinded with a randomized numbering system using a custom-built blinding script developed by Thomas Khan, then scored based on the following criteria: no pruning (Class I); partially pruned (Class II); fully pruned (Class III); and regrown (Class IV). Examples for each class are shown in FIG. 5A as the wild type images for the 6 hours after pupation (Class I), 12 hours (Class III), and 18 hours (Class IV), as well as the Fmr1 image for 12 hours (Class II). Samples were un-blinded and results recorded. The percentages in each category were calculated for each genotype at each time point; average percentages were then calculated with three independent trials. Statistical significance was determined using unpaired, two-tailed t-tests.

### Example 2—Fmr1 Mutant Flies were More Sensitive to Infection than Wild Type Flies

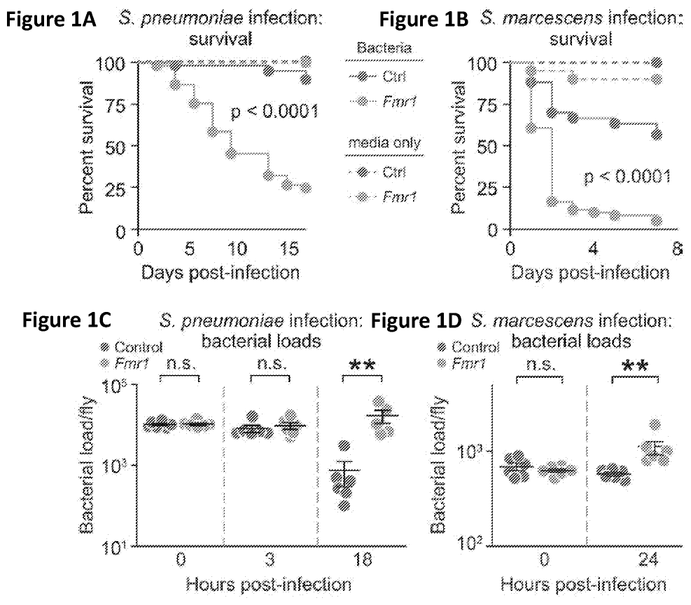

To investigate immune system function in Fmr1 mutants, animal survival in response to infection with bacterial pathogens was analyzed and it was found that Fmr1 mutants were more sensitive to infection with Streptococcus pneumoniae (FIG. 1A, p<0.0001) or Serratia marcescens (FIG. 1B, p<0.0001) compared to wild type. In both cases, Fmr1 mutants had significantly higher bacterial loads relative to wild type 18 hours after infection (FIG. 1C, p<0.01, FIG. 1D, p<0.01). This result showed that Fmr1 mutants were less able to kill and clear these two bacterial pathogens.

Antimicrobial peptides (AMPs) Drosomycin and Diptericin, canonical outputs of the immune signaling Toll and imd pathways constitute well-characterized general mechanisms of immune defense in Drosophila. The levels of Drosomycin and Diptericin expression induced by infection were similar in mutants and wild type (results not shown), showing that Fmr1 mutants were defective in an immune mechanism for killing bacteria other than AMP synthesis. Furthermore, Fmr1 mutants exhibited no differences in bacterial load after infection with two other bacterial pathogens, P. aeruginosa and L. monocytogenes (results not shown). These results together showed that Fmr1 mutants have an immune system defect that specifically compromises the clearance of S. pneumoniae and S. marcescens.

### Example 3—Fmr1 is Required for Phagocytosis of Bacteria by Hemocytes

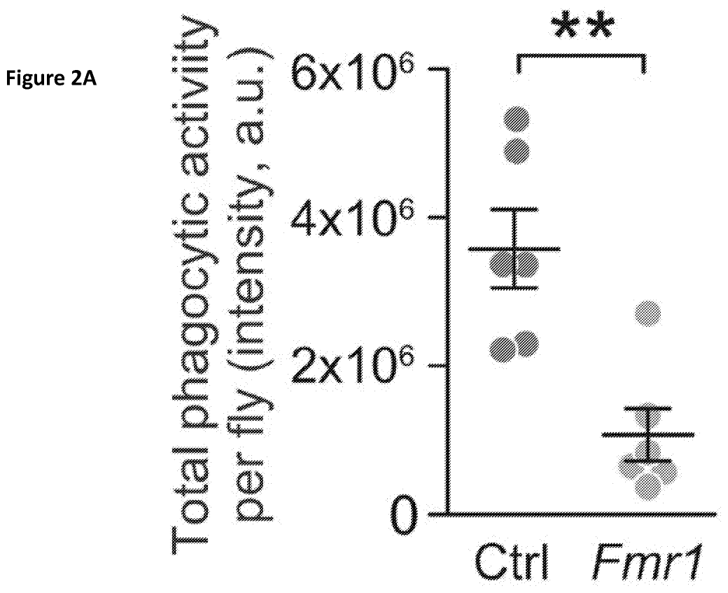

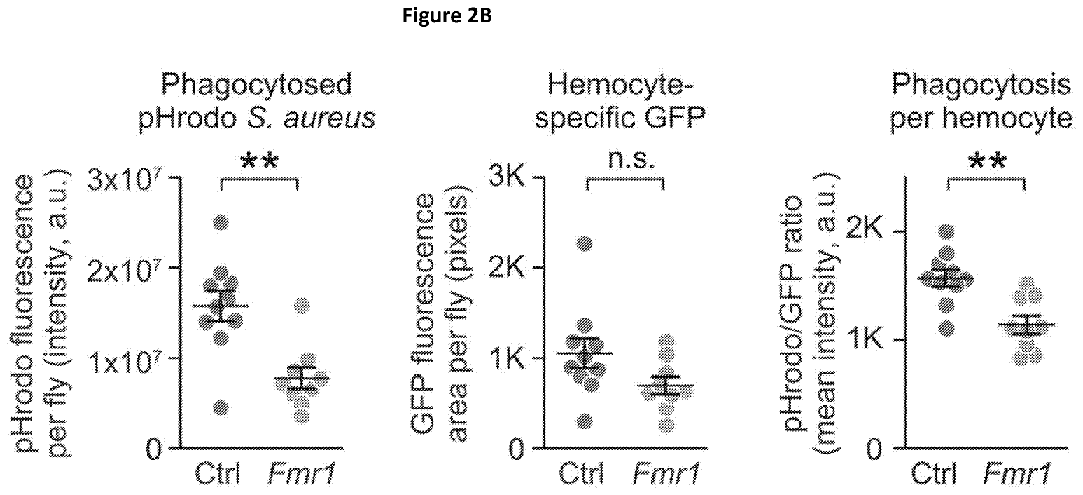

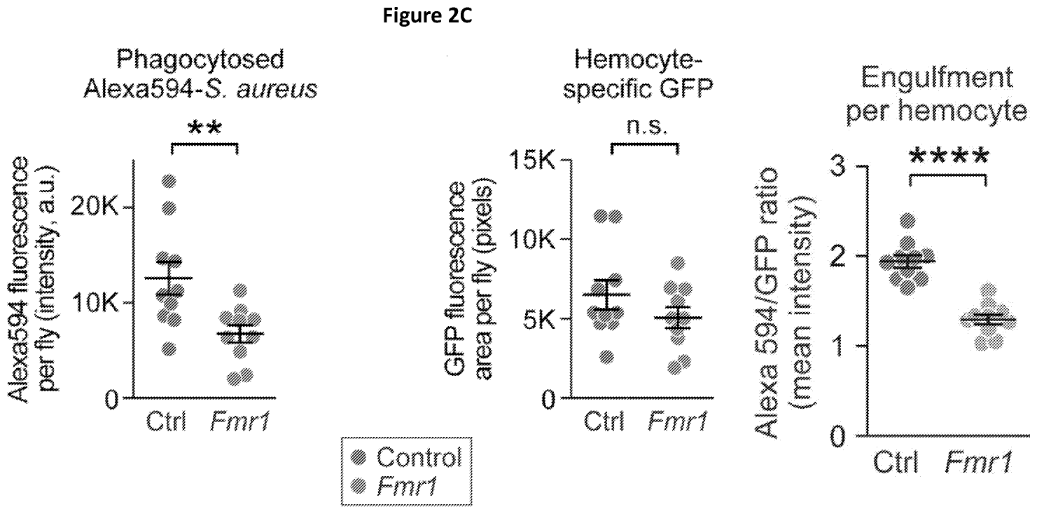

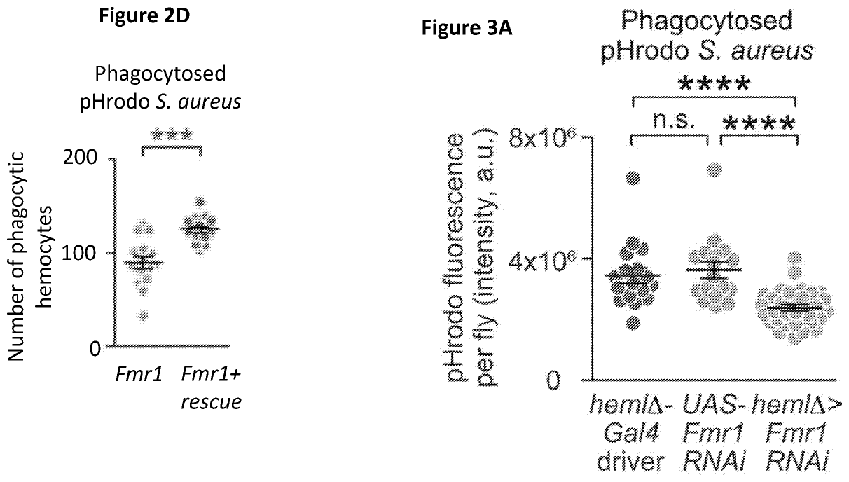

In Drosophila, reduction of bacterial load and survival of infection by S. pneumoniae or S. marcescens require phagocytosis by systemic immune cells or hemocytes (Stone et al., 2012). Phagocytosis involves bacterial engulfment into a vesicle called the phagosome, which fuses with endosomes and eventually the acidic lysosome. To directly assay the acidic lysosome step of phagocytosis, Fmr1 mutants and isogenic controls were injected with heat-inactivated Staphylococcus aureus bacteria labeled with a pH-sensitive dye (pHrodo). pHrodo fluorescence increases in acidic environments, and quantification of this signal in phagocytic hemocytes allows quantification of the number of bacteria incorporated into the lysosomal compartment. It was found that Fmr1 mutants exhibited reduced pHrodo fluorescence in hemocytes compared to wild type controls, indicating decreased phagocytic activity (p<0.01, FIG. 2A).

To determine whether this decrease in bacterial engulfment results from decreased phagocytic activity per phagocyte or a decrease in the number of phagocytes, hemocytyes were genetically labeled with GFP using a hemocyte-specific expression driver (FIG. 2B). Fmr1 mutants again had lower levels of phagocytosis than control flies (p<0.01) and also exhibited similar levels of hemocyte GFP expression in the field of view (p>0.05). Thus when pHrodo fluorescence was normalized to hemocyte GFP expression, Fmr1 mutants had significantly lower phagocytic activity per hemocyte than controls (p<0.01).

To distinguish whether phagocytosis is disrupted in Fmr1 mutants at the late step of lysosome acidification or at the earlier step of bacterial engulfment, this phagocytosis assay was performed with Alexa594-labeled S. aureus. The fluorescence signal from extracellular, nonengulfed bacteria was quenched with Trypan Blue, resulting in fluorescence emission only from intracellular bacteria. It was found that Fmr1 mutants exhibited decreased intracellular Alexa594 fluorescence relative to wild type controls, indicating that the loss of Fmr1 resulted in an early stage defect in bacterial engulfment (FIG. 2C). To confirm that this defect in phagocytosis is due to loss of Fmr1, Fmr1 null mutants with Fmr1 null mutants containing a transgenic genomic rescue construct of Fmr1 driven by its endogenous promoter were compared (FIG. 2D). It was found that this transgenic genomic rescue construct was sufficient to increase phagocytic activity.

Taken together, these results demonstrated that Fmr1 was required for phagocytosis of bacteria by hemocytes.

### Example 4—Fmr1 Plays a Cell-Autonomous Function in Phagocytosis by Hemocytes

Fmr1 is expressed ubiquitously throughout the body and hemocyte function is regulated by many extracellular factors, such as enzymes that process bacteria products and soluble signals from other tissues. To test whether Fmr1 plays a cell-autonomous role in phagocytosis by hemocytes, a hemocyte-specific Fmr1 knockdown by RNAi was performed. It was found that flies in which the hemocyte-specific hemlΔ-Gal4 expression driver was combined with a UAS-RNAi construct against Fmr1 exhibited less phagocytic activity than flies containing either the hemlΔ-Gal4 driver or RNAi construct alone (FIG. 3A). Consistent with the results for Fmr1 null mutants, when hemocytes were genetically labeled by GFP expression, it was found that hemocyte-specific knockdown of Fmr1 did not alter the number of hemocytes (FIG. 3B). Moreover, similar to Fmr1 null mutants, hemocyte-specific knockdown of Fmr1 caused sensitivity to infection by S. pneumonie (FIG. 3C). Thus, RNAi-mediated knockdown of Fmr1 specifically in hemocytes did not alter the number of hemocytes but caused a defect in their phagocytic activity that resulted in defective immunity, indicating that Fmr1 plays a cell-autonomous function in phagocytosis by hemocytes.

### Example 5—Fmr1 Mutants have a Defect in Glia-Mediated Phagocytosis

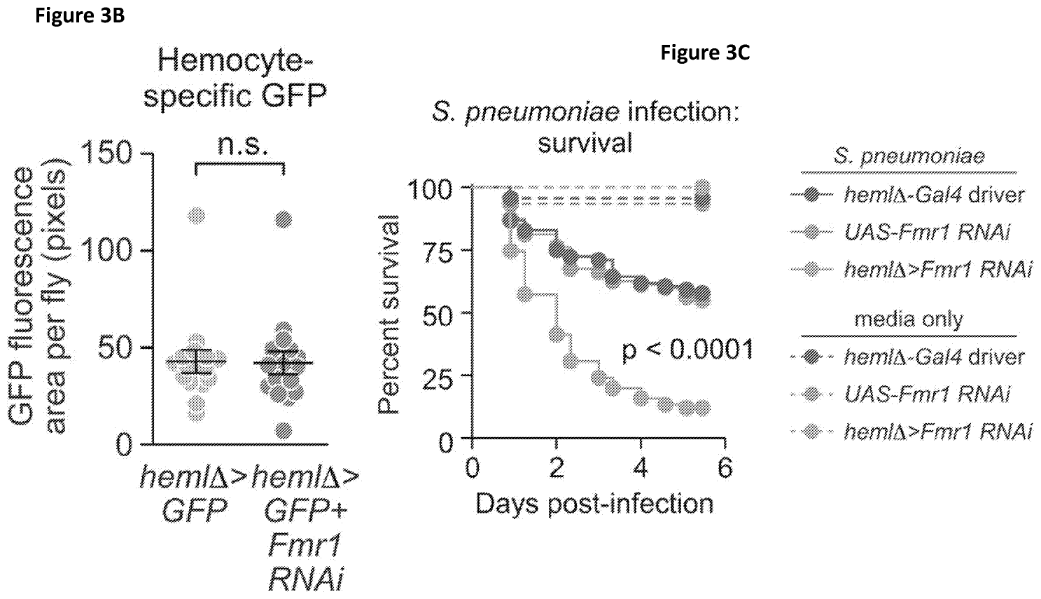

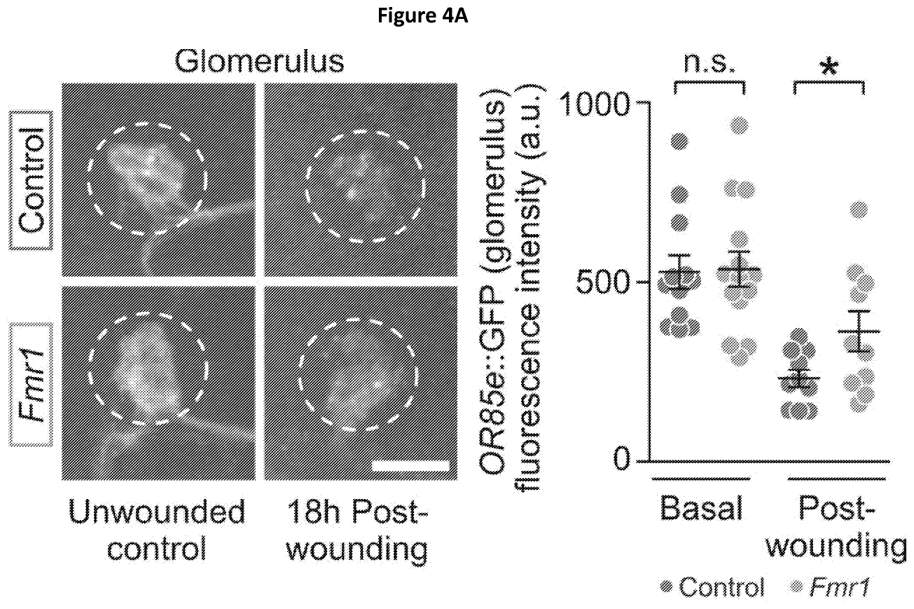

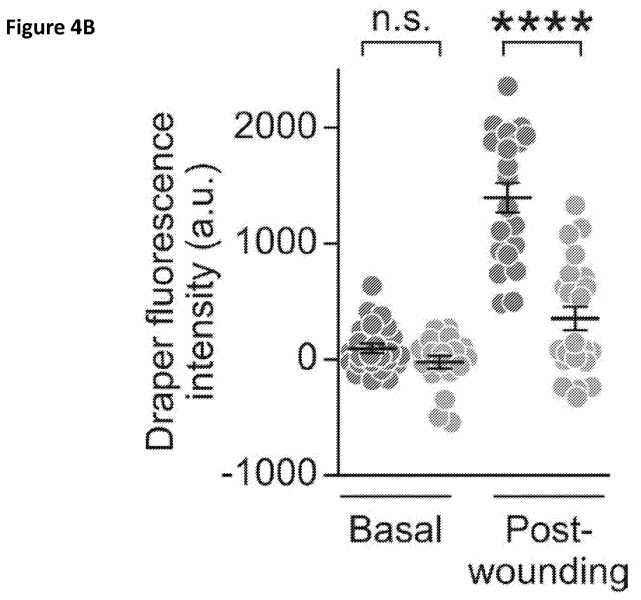

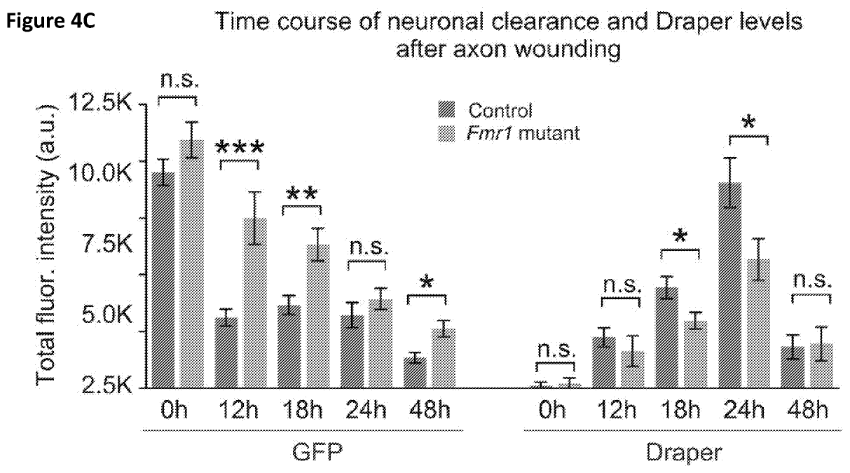

Because the cellular process of phagocytosis relies on many common molecular components in different cell types, it was determined whether in Fmr1 mutants, immune cells in the brain or glia also exhibit defects in phagocytosis. A neuronal severing (or axotomy) assay was used to monitor the glia-mediated clearance of neuronal debris in adult animals (MacDonald et al., 2006) (FIG. 4). In this assay, the cell-type specific OR85e::GFP marker was used to label a subset of olfactory receptor neurons that extend axons deep into the brain and synapse on olfactory glomeruli. When these axons are severed, the axonal remnants emit an “eat me” signal that activates a subset of phagocytic glia, termed ensheathing glia (Doherty et al., 2009). Activated glia upregulate expression of the conserved phagocytic receptor Draper and extend membranous processes to the glomeruli to phagocytose neuronal debris (Ziegenfuss et al., 2008; Ziegenfuss et al., 2012). Clearance of the GFP-labeled neuronal remnants can be quantitatively monitored by loss of GFP signal in the glomeruli (MacDonald et al., 2006). In control animals, the GFP signal from severed neurons was strongly reduced 18 hours after axotomy. In contrast, in Fmr1 mutants, the GFP signal persisted at significantly higher levels at 18 hours after axotomy (FIG. 4A, p<0.05). The volume of glomeruli was not significantly different between Fmr1 mutants and controls in unwounded animals (results not shown).

Taken together, these results indicated that Fmr1 mutants displayed defects in the clearance of neuronal remnants after axotomy and showed that these mutants have a defect in glia-mediated phagocytosis.

### Example 6—Fmr1 Mutant Adults Exhibited Defects in Recruitment of Activated Phagocytic Glia

Neuronal clearance is dependent on activation and extension of phagocytic glia to the glomeruli. To determine whether there is a defect in recruitment of phagocytic glial extensions, the localization of Draper-expressing glia in the region of the glomeruli was examined. Draper, a transmembrane receptor protein of the CED-1 family, is a marker for activated glia that is upregulated in response to neuronal injury (Doherty et al., 2014; Ziegenfuss et al., 2012).

Both Fmr1 mutants and controls exhibited low levels of Draper protein in the glomeruli of unwounded animals (results not shown). In control animals, a robust upregulation of the number of Draper expressing glia in the glomeruli was observed 18 hours after axotomy. In contrast, Fmr1 mutants exhibited significantly lower levels of Draper-expressing glia in the glomeruli at this time point (FIG. 4B, p<0.0001). A time course of neuronal clearance (neurons marked by GFP) and Draper expression in Fmr1 mutants and control animals was performed at 0, 12, 18, 24, and 48 hours post-axotomy. The reduced recruitment of activated glia to sites of neuronal damage in Fmr1 mutants produced a delay but not a complete inhibition of neuronal clearance (FIG. 4C). Though activation of Draper is thought to be required for recruitment of glia to the site of wounding, it is possible that glial recruitment is normal but these glia do not express Draper normally in Fmr1 mutants. However, upon testing, the ratio of Draper to RFP fluorescence was similar between Fmr1 mutants and control animals post-axotomy, indicating that Draper expression was not impaired in these glia of Fmr1 mutants (results not shown). That is, the decrease in Draper levels at the glomeruli in Fmr1 mutants was not due to decreased Draper expression per glia but due to decreased numbers of activated phagocytic glia at the glomeruli.

Taken together, these results demonstrated that Fmr1 mutant adults exhibited defects in recruitment of activated phagocytic glia that lead to delayed neuronal clearance after axotomy.

### Example 7—Glia-Mediated Phagocytosis was Disrupted During Development in Fmr1 Mutant Flies

Because Fragile X syndrome is a neurodevelopmental disease, it was investigated whether glia-mediated phagocytosis was also disrupted during development in Fmr1 mutants. One stage of neurodevelopment that is dependent on glia-mediated phagocytosis is the pruning of gamma neurons of the Drosophila mushroom body (γ-MB), a brain structure important for learning and memory (Tasdemir-Yilmaz and Freeman, 2014). γ-MB neurons were rapidly pruned in wild type animals immediately following pupation, with a peak in pruning typically occurring between 6 and 18 hours after pupal formation (APF) and followed by rapid regrowth of a lobe neurons (Awasaki et al., 2011; Tasdemir-Yilmaz and Freeman, 2014). The pruning of γ-MB axons in wild type and Fmr1 mutants was monitored over time by immunostaining with anti-Fasciclin II antibodies. Consistent with previous work, a stereotyped developmental pattern in wild type animals by immunostaining with anti-Fasciclin II antibodies was observed. (FIG. 5A). As described in Example 1, after images were blinded using custom software, the extent of pruning was graded by assignment to one of four morphological classes (examples shown in FIG. 5A). Although Fmr1 mutants showed similar morphology of FasII-positive γ-MB neurons at 6 hours after puparium formation (APF), Fmr1 mutants exhibited a significant delay in pruning (FIGS. 5A and 5B). At 12 hours APF, approximately 75% of wild-type brains exhibited full pruning of FasII-positive γ-MB neurons. In contrast, only approximately 30% of Fmr1 brains exhibited full pruning (FIG. 4C, p=0.01). By 24 hours APF, both wild type and Fmr1 mutants exhibited similar levels of FasII-positive γ-MB neuronal pruning and FasII-positive α lobe regrowth.

These results showed that, similar to neuronal clearance after axotomy in adults, development of the mushroom body, a second process that is dependent on glia-mediated phagocytosis, was significantly delayed in Fmr1 mutants.

### Example 8—Fmr1 Knock Our Mice have a Defect in Glial-Dependent Synaptic Pruning

Because of its accessibility, relative simplicity, and stereotyped phases of synapse formation and refinement, the mouse retinogeniculate system is a robust, well-established system to study glial-dependent synaptic pruning.

Fmr1 knockout mice (Bakker et al., 1994) and wild type controls were injected with two different autograde tracers, cholera toxin B-subunit conjugated to Alexa488 (CTB-488) and Alexa594 (CTB-594), one in each eye. The dyes are taken up by the neurons that project into the dorsal lateral geniculate (dGLN) in the brain. During postnatal development, retinal ganglion cells (RGCs) extend from each eye to the dorsal lateral geniculate nucleus (dLGN) of the brain, forming excessive ipsilateral (same side) and contralateral (opposite side) synapses that overlap. These synapses are then refined so that by postnatal day 10 (P10), each dLGN neuron receives input from a single eye, either ipsilateral or contralateral. By injecting a single tracer into each eye, the synaptic overlap in the dLGN can be visualized, and synaptic refinement quantified as loss of this overlap. Using a standard protocol (Rebsam et al., 2012; Schafer et al., 2012; Chung et al., 2013), 24-36 hours after dye injection, a transcardial perfusion was performed, and 100-uM thick vibratome sectioning of the whole dLGN. The percent of ipsilateral segregation was then quantified as the loss of overlap in ipsilateral and contralateral labeling after fluorescent microscopy. This synaptic refinement has been shown to require glia-mediated phagocytosis. Thus, a defect in synapse refinement presents as an increase in the number of synaptic processes and overlapping synaptic targeting and represents a defect in glia-mediated phagocytosis.

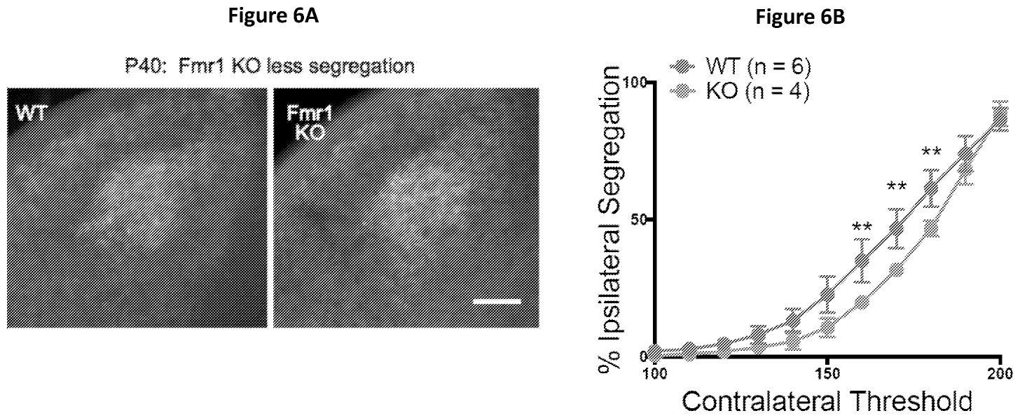

As shown in FIG. 6, at postnatal day 40 (young adult mice), a time point when such refinement is usually complete, Fmr1 knock out mice exhibited significantly decreased synapse refinement relative to wild type littermates.

These data show in vivo evidence of a synaptic elimination decrease in a mammalian model of Fragile X syndrome, defects which are consistent with glia-mediated phagocytosis defects.

### REFERENCES

Awasaki et al. 2011. Glia instruct developmental neuronal remodeling through TGF-beta signaling. Nature Neuroscience. 14:821-823.
Bakker et al. 1994 Fmr1 knockout mice: a model to study fragile x mental retardation. The Dutch-Belgium Fragile X Consortium. Cell. 78(1):23-33.
Bailey et al., (1996) J Child Psychol Psychiatry 37(1):89-126.
Bear et al. 2004. The mGluR theory of fragile X mental retardation. Trends in neurosciences. 27:370-377.
Blank and Prinz. 2012. Microglia as modulators of cognition and neuropsychiatric disorders. Glia. 61:62-70.
Chang et al. 2008. Identification of small molecules rescuing fragile X syndrome phenotypes in Drosophila. Nature Chemical Biology. 4:256-263.
Chung and Barres. 2012. The role of glial cells in synapse elimination. Current Opinion in Neurobiology. 22:438-445.
Chung et al. 2013. Astrocytes mediate synapse elimination through MEGF10 and MERKT pathways. Nature. 504:394-400.
Coffee et al. 2010. Fragile X mental retardation protein has a unique, evolutionarily conserved neuronal function not shared with FXR1P or FXR2P. Disease Models & Mechanisms. 3:471-485.
Darnell et al. 2011. FMRP stalls ribosomal translocation on mRNAs linked to synaptic function and autism. Cell. 146:247-261.
Doherty et al. 2009. Ensheathing glia function as phagocytes in the adult Drosophila brain. The Journal of Neuroscience: the Official Journal of the Society for Neuroscience. 29:4768-4781.
Doherty et al. 2014. PI3K signaling and Stat92E converge to modulate glial responsiveness to axonal injury. PLoS Biol. 12:e1001985.
Estes and McAllister. 2015. Immune mediators in the brain and peripheral tissues in autism spectrum disorder. Nature reviews. Neuroscience. 16:469-486.
Flyg and Xanthopoulos. 1983. Insect Pathogenic Properties of Serratia marcescens. Passive and Active Resistance to Insect Immunity Studied with Protease-deficient and Phage-resistant Mutants. Journal of General Microbiology. 129:453-464
Fombonne 2003. The prevalence of autism. JAMA 289(1): 87-9)
Freeman and Grinstein. 2014. Phagocytosis: receptors, signal integration, and the cytoskeleton. Immunol Rev. 262:193-215.
Gesundheit et al. 2013. Immunological and autoimmune considerations of Autism Spectrum Disorders. Journal of Autoimmunity. 44:1-7.
Hutsler and Zhang. 2010. Increased dendritic spine densities on cortical projection neurons in autism spectrum disorders. Brain Research. 1309:83-94.
Irwin et al. 2001. Abnormal dendritic spine characteristics in the temporal and visual cortices of patients with fragile-X syndrome: a quantitative examination. American Journal of Medical Genetics. 98:161-167.
Jackson and Haydon. 2008. Glial cell regulation of neurotransmission and behavior in Drosophila. Neuron Glia Biol. 4:11-17.
Jacobs and Doering. 2010. Astrocytes prevent abnormal neuronal development in the fragile x mouse. The Journal of Neuroscience: the Official Journal of the Society for Neuroscience. 30:4508-4514.
Jin and Warren. 2000. Understanding the molecular basis of fragile X syndrome. Human Molecular Genetics. 9:901-908.
Joyce et al. 2004. LuxS is required for persistent pneumococcal carriage and expression of virulence and biosynthesis genes. Infection and Immunity. 72:2964-2975.
Kelleher and Bear. 2008. The autistic neuron: troubled translation? Cell. 135:401-406.
Lee et al. 2003. Control of dendritic development by the Drosophila fragile X-related gene involves the small GTPase Rac1. Development. 130:5543-5552.
Lioy et al. 2011. A role for glia in the progression of Rett's syndrome. Nature. 475:497-500.
Logan et al. 2012. Negative regulation of glial engulfment activity by Draper terminates glial responses to axon injury. Nature Neuroscience. 15:722-730.
MacDonald et al. 2006. The Drosophila cell corpse engulfment receptor Draper mediates glial clearance of severed axons. Neuron. 50:869-881.
Mansfield et al. 2003. Exploration of host-pathogen interactions using Listeria monocytogenes and Drosophila melanogaster. Cell Microbiol. 5:901-911.
McBride et al. 2005. Pharmacological rescue of synaptic plasticity, courtship behavior, and mushroom body defects in a Drosophila model of fragile X syndrome. Neuron. 45:753-764.
Mead, and Ashwood. 2015. Evidence supporting an altered immune response in ASD. Immunology Letters. 163:49-55.
Pacey et al. 2015. Persistent astrocyte activation in the fragile X mouse cerebellum. Brain Behav. 5:e00400.
Pan et al. 2004. The Drosophila fragile X gene negatively regulates neuronal elaboration and synaptic differentiation. Current biology. 14:1863-1870.
Patel et al. 2013. A target cell-specific role for presynaptic Fmr1 in regulating glutamate release onto neocortical fast-spiking inhibitory neurons. The Journal of Neuroscience: the Official Journal of the Society for Neuroscience. 33:2593-2604.
Pfeiffer and Huber. 2009. The state of synapses in fragile X syndrome. Neuroscientist. 15:549-567.
Rebsam et al. 2012. Eye-specific projections of retinogeniculate axons are altered in albino mice. The Journal of Neuroscience: the Official Journal of the Society for Neuroscience. 32:4821-26.
Samsam et al. 2014. Pathophysiology of autism spectrum disorders: revisiting gastrointestinal involvement and immune imbalance. World Journal of Gastroenterology. 20:9942-9951.
Schafer and Stevens. 2013. Phagocytic glial cells: sculpting synaptic circuits in the developing nervous system. Current Opinion in Neurobiology. 23:1034-1040.
Schafer et al. 2012. Microglia sculpt postnatal neural circuits in an activity and complement-dependent manner. Neuron. 74:691-705.
Siller and Broadie. 2012. Matrix metalloproteinases and minocycline: therapeutic avenues for fragile X syndrome. Neural Plast. 2012:124548.
Stone et al. 2012. The circadian clock protein timeless regulates phagocytosis of bacteria in Drosophila. PLoS pathogens. 8:e1002445.
Suh and Jackson. 2007. Drosophila ebony activity is required in glia for the circadian regulation of locomotor activity. Neuron. 55:435-447.
Tang et al. 2014. Loss of mTOR-Dependent Macroautophagy Causes Autistic-like Synaptic Pruning Deficits. Neuron. 83:1131-1143.
Tasdemir-Yilmaz and Freeman. 2014. Astrocytes engage unique molecular programs to engulf pruned neuronal debris from distinct subsets of neurons. Genes & Development. 28:20-33.
Tessier and Broadie. 2008. Drosophila fragile X mental retardation protein developmentally regulates activity-dependent axon pruning. Development. 135:1547-1557.
Wan et al. 2000. Characterization of dFMR1, a Drosophila melanogaster homolog of the fragile X mental retardation protein. Molecular and Cellular Biology. 20:8536-8547.
Yin et al. 2017 (December 19). The Role of Microglia and Macrophages in the CNS homeostasis, autoimmunity, and cancer. Journal of Immunological Research. 2017: ID 5150678.
Zarnescu et al. 2005. Fragile X proteinfunctions with lgl and the par complex in flies and mice. Developmental Cell. 8:43-52.
Ziegenfuss et al. 2008. Draper-dependent glial phagocytic activity is mediated by Src and Syk family kinase signalling. Nature. 453:935-939.
Ziegenfuss et al. 2012. Distinct molecular pathways mediate glial activation and engulfment of axonal debris after axotomy. Nature neuroscience. 15:979-987.

1. An in vitro method of detecting autism or autism-spectrum disorder or a predisposition to autism or autism-spectrum disorder in a human subject, comprising:
a. isolating phagocytic immune cells from a sample from the subject;
b. contacting or incubating the cells with bioreactive particles, in an amount, and for a period of time, sufficient for phagocytic immune cells to phagocytose the bioreactive particles;
c. detecting the number or quantity of cells which phagocytosed the bioreactive particles after the period of time;
d. comparing the number or quantity of cells detected in step (c) to a reference value of the number or quantity of cells that phagocytosed the bioreactive particles in the same period of time; and
e. detecting autism or a predisposition to autism when the number or quantity of cells detected in step (c) is less than the reference value.

2. (canceled)

3. The method of claim 1, wherein an age of the human subject is chosen from the group consisting of three years old or younger, between three and five years old, and over five years old.

4. (canceled)

5. (canceled)

6. (canceled)

7. (canceled)

8. The method of claim 1, wherein the sample is blood.

9. The method of claim 1, wherein the bioreactive particles are chosen from the group consisting of bacteria, cells, portions of cells, cell fragments, latex beads, yeast, and Zymosan.

10. (canceled)

11. The method of claim 9, wherein the bacteria is chosen from the group consisting of E. coli, Streptococcus pneumoniae and Serratia marcescens.

12. The method of claim 9, wherein the cells are apoptotic.

13. (canceled)

14. The method of claim 1, wherein the bioreactive particles comprise a detectable label.

15. (canceled)

16. (canceled)

17. The method of claim 1, wherein the period of time ranges from at least 30 minutes to about 2 hours.

18. (canceled)

19. The method of claim 1, wherein the amount of bioreactive particles used in step (b) is about 5 mg/ml to 80 mg/ml.

20. (canceled)

21. The method of claim 1, wherein the reference value is from a healthy control subject.

22. An in vitro method of detecting autism or autism-spectrum disorder or a predisposition to autism or autism-spectrum disorder in a human subject, comprising:
a. isolating phagocytic immune cells from a sample from the subject;
b. contacting or incubating the cells with bioreactive particles, in an amount and for a period of time, sufficient for phagocytic immune cells to phagocytose the bioreactive particles;
c. detecting the number or quantity of bioreactive particles that are not phagocytosed after the period of time;
d. comparing the number or quantity of bioreactive particles detected in step (c) to a reference value of the number or quantity of bioreactive particles that are not phagocytosed in the same period of time; and
e. detecting autism or a predisposition to autism when the number or quantity of bioreactive particles detected in step (c) is greater than the reference value.

23. (canceled)

24. The method of claim 22, wherein an age of the human subject is chosen from the group consisting of three years old or younger, between three and five years old, and over five years old.

25. (canceled)

26. (canceled)

27. (canceled)

28. (canceled)

29. The method of claim 22, wherein the sample is blood.

30. The method of claim 22, wherein the bioreactive particles are chosen from the group consisting of bacteria, cells, portions of cells, cell fragments, latex beads, yeast, and Zymosan.

31. (canceled)

32. The method of claim 30, wherein the bacteria is chosen from the group consisting of E. coli, Streptococcus pneumoniae and Serratia marcescens.

33. The method of claim 30, wherein the cells are apoptotic.

34. (canceled)

35. The method of claim 30, wherein the bioreactive particles comprise a detectable label.

36. (canceled)

37. (canceled)

38. The method of claim 22, wherein the period of time ranges from at least 30 minutes to about 2 hours.

39. (canceled)

40. The method of claim 22, wherein the amount of bioreactive particles used in step (b) is about 5 mg/ml to 80 mg/ml.

41. (canceled)

42. The method of claim 22, wherein the reference value is from a healthy control subject.

43. A kit for the detection of autism or a predisposition to autism in a human subject, comprising: reagents for obtaining a sample; reagents for isolating, plating and culturing phagocytic immune cells from the sample; bioreactive particles for inducing phagocytosis; and instructions for use including the amount of bioreactive particles and time to contact or incubate the bioreactive particles with the cells, and a reference value for comparison or instructions how to obtain a reference value from a healthy control.

44.-52. (canceled)
